(19) United States
(12) Patent Application Publication
(10) Pub. No.: US 2003/0108981 A1
(43) Pub. Date: Jun. 12, 2003

(54) Colorimetric method for detection of dipicolinic acid in mailpieces

(76) Inventor: William L. Robinson JR., Baltimore, MD (US)

Correspondence Address: William L. Robinson Jr., 2517 Quantico Avenue, 2517 Quantico Avenue, Baltimore, MD, 21215, US

(21) Appl. No.: 10/006,150

(22) Filed: Dec. 10, 2001

(51) Int. Cl. C12Q 1/04; G01N 33/00

(52) U.S. Cl. 435/34; 436/96

(57) ABSTRACT

Dipicolinic acid (pyridine 2,6 dicarboxylic acid) is a major component of bacterial spores and is unique in that it has only been found in spores. Dipicolinic acid is synthesized during sporulation and is released during germination or upon hydrolysis or heating. Current methods of analysis are based upon ultraviolet or spectroscopic absorption techniques. Though accurate these methods are time consuming and laborious. This invention describes a convenient calorimetric method that utilizes the color complex formed by the interaction of ferrous iron and reducing agents with dipicolinic acid. The presence of dipicolinic acid from spores or toxins produced by vegetative bacterial agents such as ammonia or other volatile amines within a package or container upon which the indicator is mounted will cause an immediate and slowly reversible color change. Primary public use envelopes and personal protection badges or rings worn inside a room with potentially contaminated mail are specifically suggested.

### BACKGROUND OF THE INVENTION

1. Field of the Invention

The present invention relates generally to anti-terrorism, more particularly to antiterrorism measures for safeguarding the mail, and most specifically to safeguarding the mail from transmission of bacterial spores including those produced by Clostridia and Bacillus antracis, i.e. anthrax.

2. General Background

The specific problem addressed herein first came to the attention of the public in early October 2001 when the first case of mail transferred anthrax infection became known which was only several weeks after the terrorist destruction of the world trade center in New York City on Sep. 11, 2001. In the second month following this it became publicly apparent that anthrax bacteria was being sent through the U.S. Mail as confirmed by the deaths of several U.S. Postal Service (U.S.P.S.) employees from anthrax infection. Anthrax is contracted by cutaneous contact, digestion or inhalation of spores. Clostridium botulinum is primarily contracted by ingestion. Bubonic plague is caused by a bacterium of the genus Pasteurella and is contracted by both cutaneous contact and inhalation as is Mycobacterium tuberculosis. All of these bacterial biological agents have been recognized as current or potential terrorist weapons readily deployed through the mail.

Smallpox has also been recognized as a potential terrorist weapon that could also be deployed through the mail but is a virus and although it has been decades since smallpox vaccinations were administered on a large scale the vaccine is known to be quite good, i.e. relatively benign and easily administered in a single step. Smallpox vaccine was administered to six and a half million people, virtually the entire population of New York City, in the last public outbreak of smallpox in the United States in 1947 with only a handful of deaths resulting from the vaccination and fewer deaths from the outbreak. The only known vaccine for anthrax, in contrast, is considered problematic. The vaccine has been known for several decades but was only approved by the Federal Drug and Food Administration during the time discussed above in which mail borne anthrax infection became publicly known.

The anthrax vaccination currently available is publicly known to contain impurities from which side effects result. The vaccination, moreover, is not accomplished in a single step but requires four different inoculations months apart from each other and hence cannot be readily administered to anyone in a relatively brief amount of time. It is considered that vaccinations, which in principle rely upon the development of antibodies by the human body in reaction to a very small amount of the biological agent addressed, are inherently better suited to guard against viral rather than bacterial infections. Bacterial infections are typically combated with antibiotics, which are ineffective against viruses, and viruses are typically combated with vaccination. Every late fall or early winter there is typically a new strain of influenza virus for which a vaccine is developed and administered to people requesting the same.

Statement of Need

Anthrax spores are particularly virulent and lethal because by the time that infection is manifest antibiotics are relatively ineffective. Without either a vaccine which can be safely and readily administered to large numbers of people or diagnosis early enough to enable effective treatment with antibiotics it is considered that anthrax, and other similar biological agents which can be contracted by contact or inhalation, presents a very serious terrorist weapon readily deployed through the mail and therefore a poignant need exists for an effective means of preventing such infection by means of this vector.

### SUMMARY OF THE INVENTION

Objects of the Invention

A primary object of the present invention is the prevention of transmitting bacterial spores produced by biological agents including Bacillus antracis, i.e. anthrax, through the mail.

A first ancillary objective of the present invention is a system for ensuring that mail does not contain spores produced by biological agents including Bacillus antracis, i.e. anthrax.

A first auxiliary objective of the present invention is a means of recognizing mail as containing spores produced by biological agents including Bacillus antracis, i.e. anthrax, without opening that mail.

A second ancillary objective of the present invention is means of recognizing mail containing spores produced by biological agents including Bacillus antracis, i.e. anthrax, before handling that mail.

Principles Relating to the Present Invention

In achievement of the above stated objectives it is considered that packaging and containers for mail be equipped with means of indicating the presence of bacterial spores produced by biological agents including Bacillus antracis, i.e. anthrax, within that mail. It is suggested that an indicator compound which changes color, upon interaction with Dipicolinic acid produced by spores from dormant bacteria, be utilized for visual indication. The indicator compound must be in communication with the interior of the package or container but must be read from the outside without opening the package or container which must further provide an effective seal against leakage of any bacteria therein. Paper is sufficient to provide this seal if a fill enclosure is obtained and plastic, metal, and other materials are perfectly satisfactory in providing a sufficient seal if a full physical enclosure is effected.

An otherwise conventional paper envelope with a substantially transparent clear panel with an indicator disposed interiorly thereupon is suggested for primary public use. The indicator preferably is made from an indicator solution having a pH of 2-6 disposed upon an appropriately porous substrate. A polymeric matrix, particularly including alkoxysilanes, is specifically recommended which is readily manufactured in thin sheets and readily adheres to paper and other plastics including glassine, Cellophane™, and polyethylene which are specifically suggested for the clear panel. But the substrate can be paper, cotton, string, or any moderately porous material of essentially neutral color which will not obscure the change in color of the indicator solution therein caused by from exposure to Dipicolinic acid produced by bacterial spores. The change in color develops immediately but is unstable however, the addition of a reducing agent such as ascorbic acid yields a complex that is stable. The calorimetric compound contains Fe(NH₄)₂(SO₄)₂ & 6H₂O and ascorbic acid in acetate buffer at pH 5.5.

The rate of response and detection sensitivity of the indicator solution is improved by use of non-volatile acids and other components including concentrated sulfuric acid, sulfamic acid, phosphoric acid, zeolites, alumina, polyacrylic acid and suphonated perfluoroethylene. With further regard to an envelope providing a visual indication of bacterial spores contained therein it is considered that such envelopes, and all other mail packaging including boxes which are intended for use by the public in enclosing material to be mailed, further preferably possess a means of verifying authenticity which is preferably invisible to humans but machine readable. A thin film plastic with magnetically stored information is suggested as are other authenticity means commonly deployed upon currency. Electronic fingerprints or ‘watermarks’ read by scanning devices embedded in a plastic window to which the substrate is affixed is specifically recommended. The objective is to avoid fraudulent imitation of the envelope or packaging.

The foregoing is intended to provide one practiced in the art with the best known means of making and utilizing an embodiment in accordance with the principles relating to the present invention and is not to be construed in any manner as restrictive of said invention or rights and privileges secured by letters Patent for which I claim:

1. An anti-terrorist system, intended to prevent the transmission of bacterial spores produced by biological agents including Clostridia and Bacillus antracis, i.e. anthrax, and the toxins produced by said agents through the mail and to assist in the detection and identification of specific pieces of mail containing spores produced by bacterial biological agents including Clostridia and Bacillus antracis, said system comprising:
at least one bacterial biological agent/toxin indicator mounted upon a wall of a mailing enclosure adapted for use in mailing materials;
said bacterial biological agent/toxin indicator possessing an appropriately porous substrate coated with ferrous iron and a reducing agent solution which changes color in response to interaction with Dipicolinic acid;
a window made of a substantially transparent material substantially contiguous with said wall;
said appropriately porous substrate coated with a ferrous iron and reducing agent indicator solution being disposed by the mounting of said indicator upon a wall of said enclosure in communication with the interior of said mailing enclosure and interiorly adjacent said window;
whereby spores produced by dormant bacterial biological agents including Clostridia and Bacillus antracis contained inside said mailing enclosure are detected by observing, through said window, the change in color of the ferrous iron and reducing agent solution coating upon said appropriately porous substrate resulting from contact with the Dipicolinic acid.

2. An anti-terrorist system in accordance with claim 1 utilizing an indicator solution containing ferrous iron or a derivative and a reducing agent such as ascorbic acid coated upon said appropriately porous substrate whereupon without aqueous mediation said bacterial biological spore indicator is operable below the freezing point of water.

3. An anti-terrorist system in accordance with claim 1 wherein the rate of response and detection sensitivity of the indicator is improved by use of non-volatile acids and other components including concentrated sulfuric acid, sulfamic acid, phosphoric acid, zeolites, alumina, polyacrylic acid and suphonated perfluoroethylene.

4. An anti-terrorist system in accordance with claim 1 utilizing a window made of substantially transparent sheet material disposed over an aperture through said wall.

5. An anti-terrorist system in accordance with claim 4 utilizing paper in construction of said envelope.

6. An anti-terrorist system in accordance with claim 4 having said porous substrate coated with ferrous iron and a reducing agent indicator solution disposed interiorly adjacent said window at a consistent positioning which regard to the perimeter of the envelope proximate the top edge of said envelope to facilitate reading by automatic scanning machines.

7. An anti-terrorist system in accordance with claim 6 having said appropriately porous substrate coated with ferrous iron and a reducing agent indicator solution affixed to said window made of substantially transparent sheet material.

8. An anti-terrorist system in accordance with claim 6 having an electronic fingerprint deposed thereupon by which verification of the mailing enclosure can be automatically made by scanning said electronic fingerprint.

9. An antiterrorist system, intended to prevent the transmission of vegetative bacterial biological agents including Bacillus antracis, i.e. anthrax, through the mail and to assist in the detection and identification of specific pieces of mail containing bacterial biological agents including Bacillus antracis, said system comprising:
at least one bacterial biological agent/toxin indicator mounted upon a wall of a mail enclosure adapted for use in enclosing mail;
said bacterial biological agent/toxin indicator possessing an appropriately porous substrate coated with an acidic acid-base solution which changes color in reaction to being neutralized by volatile compounds including ammonia and other volatile amines;
a window made of a substantially transparent material substantially contiguous with said wall;
said appropriately porous substrate coated with an acidic acid-base indicator solution being disposed by the mounting of said bacterial biological indicator upon a wall of said enclosure in communication with the interior of said mail enclosure and interiorly adjacent said window;
whereby vegetative bacterial biological agents including Bacillus antracis emitting gaseous amines and toxins produced by said agents inside said mail enclosure are detected by observing, through said window, the change in color of the acidic acid-base solution coating upon said appropriately porous substrate resulting from pH neutralization.

10. An anti-terroist system in accordance with claim 9 wherein said rate of response and detection sensitivity of the indicator is improved by use of non-volatile acids and other components including concentrated sulfuric acid, sulfamic acid, phosphoric acid, zeolites, alumina, polyacrylic acid and suphonated perfluoroethylene.

11. An anti-terrorist system in accordance with claim 9 utilizing a window made of a substantially rigid transparent material.

12. An anti-terrorist system in accordance with claim 9 utilizing a plug style window possessing a cavity for disposition of said appropriately porous substrate coated with said acidic acid-base indicator solution.

13. An anti-terrorist system, intended to prevent the transmission of live bacterial biological agents including Bacillus antracis, i.e. anthrax, through the mail and to assist in the detection and identification of specific pieces of mail containing bacterial biological agents including Bacillus antracis, said system comprising:
at least one bacterial biological agent/toxin indicator mounted upon a wall of an article to be worn inside an enclosure containing mail;
said bacterial biological agent/toxin indicator possessing an appropriately porous substrate coated with an acidic acid-base solution which changes color in reaction to being neutralized by ammonia or other volatile bases including amines or coated with a ferrous iron and reducing agent solution changes color upon interaction with Dipicolinic acid;
said appropriately porous substrate coated with an acidic acid-base indicator solution or coated with a ferrous iron and reducing agent solution being disposed by the mounting of said bacterial biological indicator upon a wall of said article in communication with the interior of enclosure containing mail;
whereby spores produced by dormant bacterial biological agents including Bacillus antracis or gaseous ammonia, amines and/or toxins produced by vegetative bacterial biological agents inside said enclosure containing mail are detected by observing the change in color of the ferrous iron and reducing agent or acidic acid-base solution coating upon said appropriately porous substrates resulting from pH neutralization.

14. An anti-terrorist system in accordance with claim 13 in which said article to be worn inside an enclosure containing mail consists of a badge or ring indicator which can be worn by someone in a room containing mail.

15. An anti-terrorist system in accordance with claim 13 in which said badge indicator possesses a clip which facilitates attachment to clothing worn by someone in a room containing mail.

16. An anti-terrorist system in accordance with claim 13 in which said substrate coated with ferrous iron and a reducing agent or an acidic acid-base indicator solution is affixed to said badge or ring to be worn inside an enclosure containing mail is attached by adhesive.
